(19) United States
(12) Patent Application Publication
(10) Pub. No.: US 2005/0287107 A1
(43) Pub. Date: Dec. 29, 2005

(54) Odor control device

(76) Inventor: Anthony Tuccelli, Salisbury, MA (US)

Correspondence Address: Bourque & Associates, P.A., Suite 303, 835 Hanover Street, Manchester, NH, 03104, US

(21) Appl. No.: 10/877,891

(22) Filed: Jun. 25, 2004

(51) Int. Cl. A61L 9/00; A61L 9/01

(52) U.S. Cl. 424/78.1

(57) ABSTRACT

An odor control device can be discretely worn and used to minimize the effects of an unpleasant or offensive odor. The odor control device includes a flexible support strap or band and a fragrance pad. The support strap may be sized and shaped to fit around a portion of a user's head such that the fragrance pad is disposed proximate a user's nose. Alternatively, the support strap may be sized to fit around a user's wrist. According to yet another embodiment, the support strap may include an adhesive region such that the odor device may be removably adhered to the user (preferably proximate the user's nose). The support strap may also include a breakaway portion which allows the support strap to separate once a force applied to the odor control device exceeds a predetermined threshold, thus preventing injury to the user should the odor control device be pulled on.

### TECHNICAL FIELD

The present invention relates to odor devices and more particularly, relates to an easily used and concealed odor control odor device.

### BACKGROUND INFORMATION

There are many situations where a person must cope with an offensive or unpleasant odor. For example, changing a person's diaper, cleaning a dirty bathroom, and picking up/removing trash are but a few examples of situations where unpleasant odors are common. Not only can the offensive odor be unpleasant, but the unpleasant odor can cause for some people to become dizzy, sick, and even prevent them from being in the situation all together.

A common solution is to use deodorizers, for example aerosol room fresheners and the like, to mask or cover-up the offensive odor. While these aerosol deodorizers are generally effective at masking or covering up the offensive odor, they suffer from several limitations.

One significant limitation of room deodorizers is that the use of room deodorizers is often embarrassing for others in a room. For example, spraying an aerosol room deodorizer is often very embarrassing for patients in a hospital or nursing home, many of whom are often already self-conscience about being in the hospital or nursing home in the first place. Because of the potentially embarrassing effects spraying an aerosol room deodorizers can cause to patients, many hospitals and nursing homes have policies against their use.

Another limitation of room deodorizers is that some people are allergic to them. While the symptoms vary dramatically from person to person, room deodorizers can cause some people to cough, become dizzy, lose their appetite, and even cause difficulty breathing. Additionally, some people find the scent of the room deodorizers themselves to be offensive smelling. This is particularly problematic for patients in a hospital or nursing home whom must spend a considerable amount of time in the room afterwards.

Moreover, the heating and air conditioning systems of many hospitals, nursing homes, and other buildings often re-circulate a portion of the air inside a room. Consequently, a room deodorizer applied in one room will often be re-circulated into other rooms, further complicating their use.

Yet another limitation of room deodorizers is that they are not very effective outdoors. For example, strong air currents often make the use of aerosol deodorizers ineffective outdoors since the deodorizer will be carried away or diluted very quickly. Additionally, the shear size of the area can make the use of aerosol deodorizers futile, ineffective, and expensive.

Accordingly, what is needed is a device that reduces the effect of an offensive odor. The device should preferably be inconspicuous so as to reduce or minimize potential embarrassment of others. Additionally, the device should preferably reduce or minimize potential allergic reactions of others. The device should preferably function both indoor as well as outdoors.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention, which is not to be limited except by the following claims.

### SUMMARY

According to one embodiment, the present invention features an odor control device having at least one flexible, stretchable support strap supporting at least one fragrance pad adapted to contain at least one fragrance. According to one embodiment, the support strap is sized and shaped to fit around at least a portion of a user's head such that the fragrance pad is disposed proximate a user's nose in a first position and is disposed proximate a user's neck in a second position.

The support strap may also include a first and a second support strap. The first support strap is preferably sized and shaped to fit above the ears of the user and around a portion of the user's head. The second support strap is preferably sized and shaped to fit around a portion of the user's head beneath the user's ears. Alternatively, the support strap may be sized and shaped to fit around a user's wrist or may include an adhesive region adapted to removably secure the odor control device to the user.

Optionally, the support strap may include a breakaway portion. The breakaway portion reduces the likelihood of injury by allowing the stretchable support strap to separate when a force applied against the odor control device exceeds a predetermined threshold.

The fragrance pad may include a material adapted to absorb a quantity of a liquid fragrance. Alternatively, the fragrance pad may include a material impregnated with a fragrance. The fragrance pad may include a generally rectangular shaped sized and shaped to fit proximate a user's nose or may be contoured to fit about a portion of a user's nose. Optionally, the odor control device may include a cover adapted to be disposed about at least a portion of the at least one fragrance pad.

### BRIEF DESCRIPTION OF THE DRAWINGS

These and other features and advantages of the present invention will be better understood by reading the following detailed description, taken together with the drawings wherein:

FIG. 1 is a plan view of one embodiment of the odor control device according to the present invention;

FIG. 2a is a front plan view of one embodiment of the odor control device shown in use on a user according to the present invention;

FIG. 2b is a side plan view of one embodiment of the odor control device shown in use on a user according to the present invention;

FIG. 3a is a front plan view of another embodiment of the odor control device shown in use on a user according to the present invention;

FIG. 3b is a side plan view of another embodiment of the odor control device shown in use on a user according to the present invention;

FIG. 4 is a front plan view of the odor control device shown in FIG. 1 on a user while not being used according to the present invention;

FIG. 5 is a plan view of yet another embodiment of the odor control device user according to the present invention; and

FIG. 5 is a plan view of a further embodiment of the odor control device user according to the present invention.

### DETAILED DESCRIPTION OF THE PREFERRED EMBODIMENTS

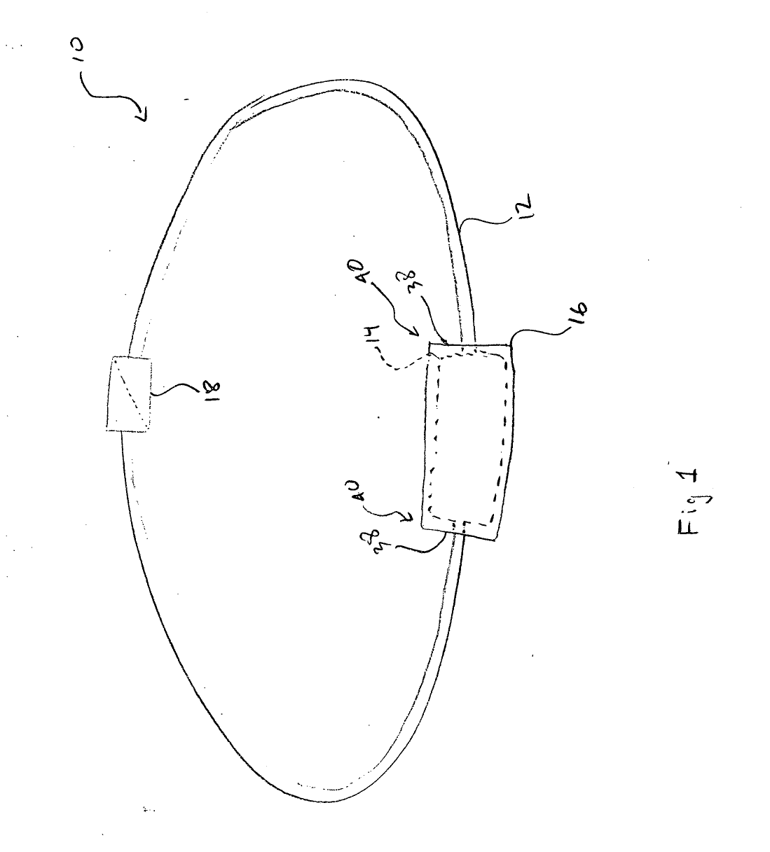

An odor control device 10, FIG. 1, according to the present invention, minimizes the negative effects of an offensive or unpleasant odor by allowing the user to inhale or breathe a pleasantly smelling fragrance/deodorant. As will be described in greater detail hereinbelow, the odor control device 10 may be discretely worn and used only when needed, thus minimizing the potentially embarrassing effects. Moreover, the likelihood of allergic reactions to the fragrance contained on the odor control device 10 is minimized since the fragrance is contained only on the fragrance pad 14. Additionally, because fragrance is contained on the fragrance pad 14, the odor control device 10 may be used indoor as well as outdoors, and even in areas with high air currents.

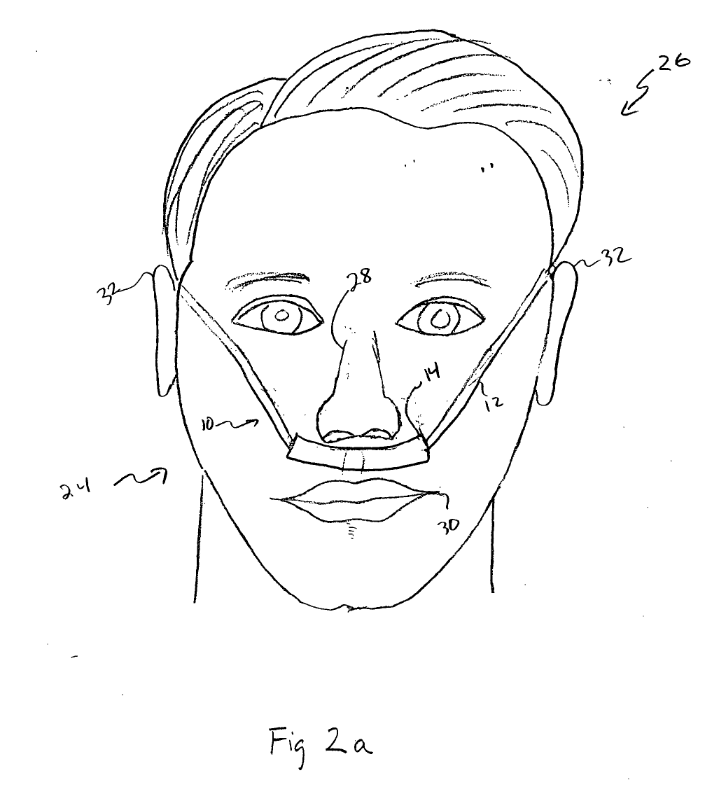

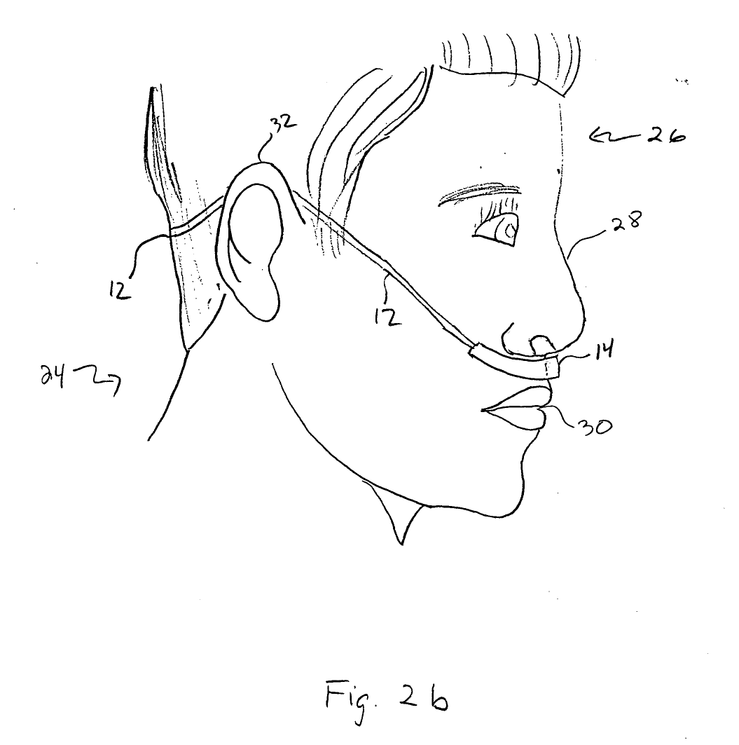

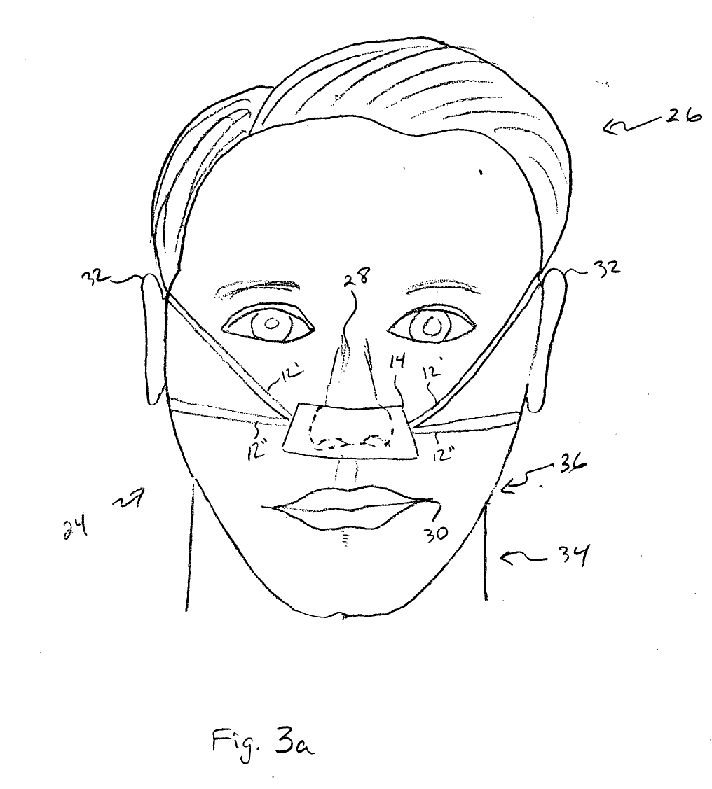

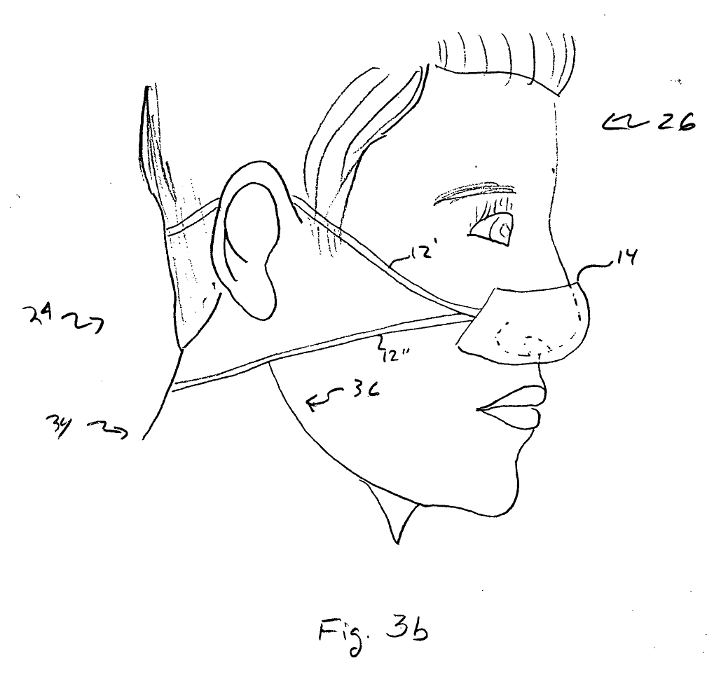

According to one embodiment, the odor control device 10 includes a flexible support strap 12 and a fragrance pad 14. The fragrance pad 14, FIGS. 2-3, is preferably sized and shaped to fit proximate the nose 28 of the user 26 while in use. According to the preferred embodiment, the fragrance pad 14 is contoured to fit around at least a portion of the nose 28 of the user. Alternatively, the fragrance pad 14 may also include a generally rectangular shaped and may also be disposed beneath or beside the nose 28 of the user 26. In any event, the fragrance pad 14 should be disposed close enough to the user's 26 nose 28 or mouth 30 such that as the user breathes (either through the nose 28 or mouth 30), the user will inhale some of the fragrance contained on the fragrance pad 14.

The fragrance pad 14 may include any material known to those skilled in the art capable of releasing an odor or fragrance. In the preferred embodiment, the fragrance pad 14 includes an absorbent material such as, but not limited to, cotton, felt, or the like, which absorbs a liquid fragrance. This embodiment is particularly useful since the fragrance pad 14 may be “recharged” with more fragrance should the strength of the fragrance diminish over time. Also, this embodiment allows the user to pick or change the scent of the fragrance. Alternatively, the fragrance pad 14 may include an impregnated material such as, but not limited to, plastic or the like.

The odor control device 10, FIGS. 2-4, includes at least one flexible support strap 12 adapted to contain or support the fragrance pad 14. According to one embodiment, the support strap 12 is sized and shaped to fit around at least a portion of the head 24 of a user 26. In the preferred embodiment, the support strap 12 is made from a stretchable or elastic material such as, but not limited to, neoprene, spandex, rubber, nylon, and the like.

The support strap 12, FIG. 1, optionally includes a breakaway portion 18 that prevents or reduces injury to the user 26 in the event that a patient pulls on the odor control device 10 or the odor control device 10 becomes caught-up on something. While the support strap 12 is preferably made from a stretchable or deformable material, the breakaway portion 18 adds additional safety to the odor control device 10 by separating the support strap 12 once a force on the odor control device exceeds a predetermined threshold. In the preferred embodiment, the breakaway portion 18 includes a perforated section. Alternatively, the breakaway portion 18 may also include a segment of easily broken or weak material, hooks and loops, or the like.

As shown in FIG. 2, while in use the support strap 12 of the odor control device 10 is preferably sized and shaped such that the support strap 12 goes over the user's 26 ears 32 and around the user's 26 head 24 and the fragrance pad 14 is held proximate the nose 28 or mouth 30 of the user 26. An advantage of this arrangement is that it simplifies the use of the odor control device 10 by making the removal and placement of the odor control device 10 easily and quickly accomplished with only one hand. Also, the simplicity of the construction allows the odor control device 10 to be inexpensively manufactured.

Alternatively, as shown in FIG. 3, the support strap 12 may include a first 12′ and at least a second 12″ support strap. The first support strap 12′ is preferably sized and shaped to fit over the user's 26 ears 32 and around the user's 26 head 24 as described above. The second strap 12″ is preferably sized and shaped to fit beneath the user's 26 ears 32 (for example proximate a user's 26 neck 34 or chin 36). The supports straps 12′, 12″ hold the fragrance pad 14 proximate the nose 28 or mouth 30 of the user 26.

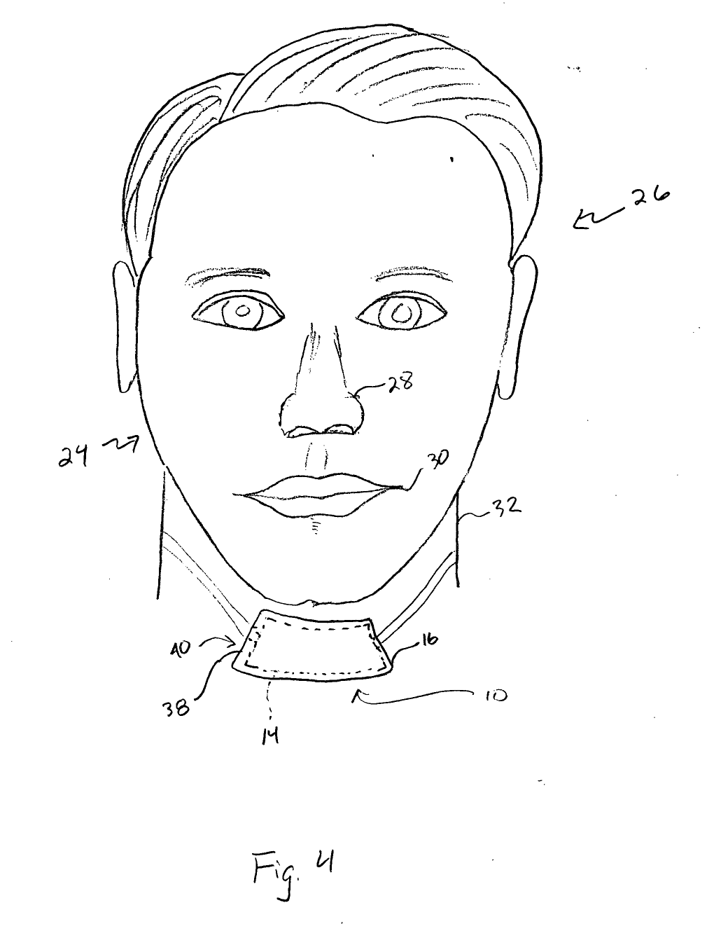

When the odor control device 10, FIG. 4, is not in use, the odor control device 10 may be disposed proximate the neck 32 of the user 26. In this manner, the odor control device 10 may be hidden from view by the user's cloths, but yet is readily accessible.

In the preferred embodiment, the odor control device 10 also includes a removable cover that is preferably sized and shaped to fit around at least a portion of the fragrance pad 14. The removable cover 16 prevents or reduces the fragrance pad 14 from continuously emitting, evaporating, and wasting the fragrance while the odor control device 10 is not in use. In the exemplary embodiment, the removable cover 16 includes a slip-over type cover, preferably constructed from plastic or the like. Optionally, the removable cover 16 may include a sealing device 38 proximate one or more of the ends 40 of the cover 16. The sealing device 40 includes any device known to those skilled in the art of reducing the amount of fragrance lost from the fragrance pad 14 including, but not limited to, hooks and loops, an elastic or stretchable cover, or an adhesive.

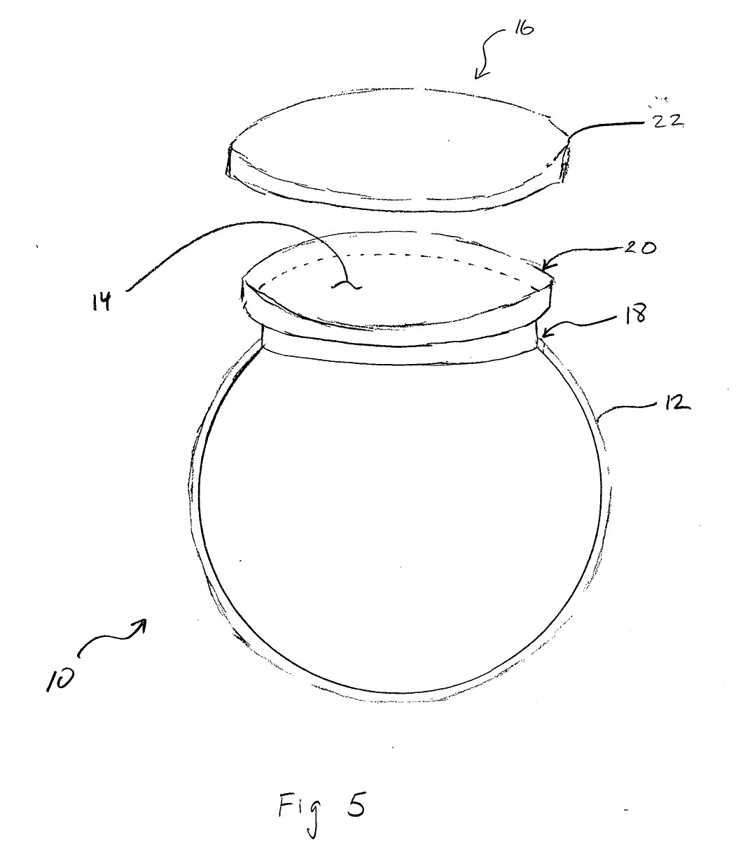

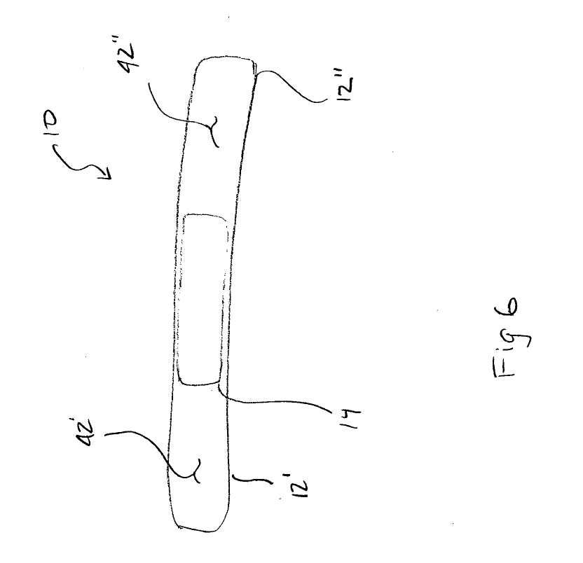

According to another embodiment, the odor control device 10, FIG. 5, includes at least one container 20 having at least one fragrance pad 14 and a support strap or band 12 sized and shaped to fit around the wrist of a user. In use, the user inhales the fragrance from the fragrance pad 14 and then holds his/her breath, thus reducing the effects of the offensive odor. This embodiment is particularly useful in situations where it is of utmost importance to minimize the potentially embarrassing situation or where it is likely that the odor control device 10 may be pulled on it worn around a user's neck/head.

The support strap 12 is preferably made from a stretchable or elastic type material as described above. The odor control device 10 may also include removable cover 16. In the preferred embodiment, the removable cover 16 includes a removable lid 22 sized and shaped to fit around at least a portion of the container 20. Optionally, the odor control device 10 may include a breakaway portion 18 as described above.

In yet another embodiment, the odor control device 10, FIG. 6, includes a fragrance pad 14 as described above dispose on a support strap 12 having an adhesive region 42 on at least one surface of the support strap 12. The support strap 12 is preferably elastic as described above and preferably includes a first 12′ and at least a second 12Δ support strap each having an adhesive region 42. The adhesive region 42 preferably includes any type of adhesive known to those skilled in the art that is compatible with human skin. The odor control device 10 is preferably placed proximate the nose or mouth of the user and is removably held in place with the adhesive regions 42. Alternatively, the odor control device 10 may be placed proximate a user's wrist and the user may inhale the fragrance from the fragrance pad 14 and then hold their breath.

As mentioned above, the present invention is not intended to be limited to a system or method which must satisfy one or more of any stated or implied object or feature of the invention and should not be limited to the preferred, exemplary, or primary embodiment(s) described herein. The foregoing description of a preferred embodiment of the invention has been presented for purposes of illustration and description. It is not intended to be exhaustive or to limit the invention to the precise-form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as is suited to the particular use contemplated. All such modifications and variations are within the scope of the invention as determined by the claims when interpreted in accordance with breadth to which they are fairly, legally and equitably entitled.

1. An odor control device comprising:
at least one flexible, stretchable support strap; and
at least one fragrance pad dispose on said at least one flexible, stretchable support strap, said fragrance pad adapted to contain at least one fragrance.

2. The odor control device as claimed in claim 1 wherein said flexible, stretchable support strap is sized and shaped to fit around at least a portion of a head of a user.

3. The odor control device as claimed in claim 2 wherein said flexible, stretchable support strap includes a breakaway portion, wherein said breakaway portion causes said flexible, stretchable support strap to separate when a force applied against said odor control device exceeds a predetermined threshold.

4. The odor control device as claimed in claim 2 further including a cover adapted to be disposed about at least a portion of said at least one fragrance pad.

5. The odor control device as claimed in claim 2 wherein said fragrance pad includes a material adapted to absorb a quantity of a liquid fragrance.

6. The odor control device as claimed in claim 2 wherein said fragrance pad includes a material impregnated with a quantity of a fragrance.

7. The odor control device as claimed in claim 2 wherein said fragrance pad includes a generally rectangular shaped sized and shaped to fit proximate a user's nose.

8. The odor control device as claimed in claim 2 wherein said fragrance pad is contoured to fit about at least a portion of a user's nose.

9. The odor control device as claimed in claim 2 wherein said flexible, stretchable support strap further includes:
a first support strap sized and shaped to fit above the ears of said user and around a portion of said user's head; and
a second support strap sized and shaped to fit around a portion of said user's head beneath the user's ears.

10. The odor control device as claimed in claim 1 wherein said flexible, stretchable support strap is sized and shaped to fit around at least a portion of a wrist of a user.

11. The odor control device as claimed in claim 10 wherein said flexible, stretchable support strap includes a breakaway portion, wherein said breakaway portion causes said stretchable support strap to separate when a force applied against said odor control device exceeds a predetermined threshold.

12. The odor control device as claimed in claim 1 wherein said flexible, support strap includes at least one adhesive region adapted to adhere said odor control device to a user.

13. A odor control device comprising:
at least one fragrance pad adapted to absorb a quantity of at least one liquid fragrance;
a cover adapted to fit about said at least one fragrance pad; and
at least one support strap supporting said eat least one fragrance pad, said support strap forming a flexible, generally circular shape sized to fit around at least a portion of a head of a user whereby in a first position, said at least one support strap is adapted to hold said at least one fragrance pad proximate a nose of said user and whereby in a second position, said cover is disposed about at least a portion of said fragrance pad and said at least one support strap is adapted to hold said odor control device proximate a neck of said user.

14. The odor control device as claimed in claim 13 wherein said support strap includes a stretchable material.

15. The odor control device as claimed in claim 14 wherein said stretchable support strap further includes:
a first support strap sized and shaped to fit above the ears of said user and around a portion of said user's head; and
a second support strap sized and shaped to fit around a portion of said user's head beneath the user's ears.

16. The odor control device as claimed in claim 13 wherein said support strap includes a breakaway portion, wherein said breakaway portion causes said support strap to separate when a force applied against said odor control device exceeds a predetermined threshold.

17. An odor control device comprising:
at least one container, said container adapted to hold a quantity of at least one fragrance;
a cover adapted to fit about said at least one container; and
at least one band supporting said least one container, said band forming a flexible, generally circular shape sized to fit around at least a portion of a wrist of a user.

18. The odor control device as claimed in claim 17 wherein said container includes a fragrance pad adapted to absorb a quantity of at least one liquid fragrance.

19. The odor control device as claimed in claim 17 wherein said container includes a fragrance pad impregnated with a quantity of at least one fragrance.

20. The odor control device as claimed in claim 17 wherein said band includes a breakaway portion, wherein said breakaway portion causes said band to separate when a force applied against said odor control device exceeds a predetermined threshold.
